(19) United States
(12) Patent Application Publication
(10) Pub. No.: US 2018/0101001 A1
(43) Pub. Date: Apr. 12, 2018

(54) CELL SELECTING APPARATUS AND CELL SELECTING SYSTEM

(71) Applicant: Kabushiki Kaisha Toshiba, Minato-ku (JP)

(72) Inventors: Tsuyoshi HIOKI, Ota (JP); Masaki TOHYAMA, Yokohama (JP); Shinya NUNOUE, Ichikawa (JP)

(73) Assignee: Kabushiki Kaisha Toshiba, Minato-ku (JP)

(21) Appl. No.: 15/699,567

(22) Filed: Sep. 8, 2017

(30) Foreign Application Priority Data
Oct. 6, 2016 (JP) 2016-198405

(51) Int. Cl. G02B 21/36; G02B 21/00; C12M 1/00; G01N 15/14; G02B 21/18

(52) U.S. Cl. CPC G02B 21/365; G02B 21/0032; G02B 21/361; G01N 15/14; G02B 21/18; C12M 47/04

(57) ABSTRACT

A cell selecting apparatus including a light source device for placing a cell sample, the light source device including a plurality of light source elements arranged in a matrix form.

### CROSS-REFERENCE TO RELATED APPLICATIONS

This application is based upon and claims the benefit of priority from Japanese Patent Application No. 2016-198405, filed on Oct. 6, 2016, and the entire contents of which are incorporated herein by reference.

### FIELD

The present invention relates to a cell selecting apparatus and a cell selecting system.

### BACKGROUND

In the field of cell diagnosis, regenerative medicine and the like, in order to evaluate and analyze cell units, for example, it is necessary to take an operation of selecting desired cells by removing unnecessary cells from a plurality of cells in a sample container and selecting only normal cells.

Meanwhile, with the development of imaging technique and image analysis technique in recent years, it is possible to grasp the detailed state of individual cells in a container.

For example, a fluorescence activated cell sorting (FACS) utilizing fluorescence emission of cells or a method of selectively sorting cells by irradiating cells with light using a photoresponsive cell culture substrate has been developed, and it is possible to simultaneously grasp the coordinate position and state of each cell during cell culture.

However, in the FACS, the coordinate position of each cell is lost when transferring the cells from the container to the FACS device. Furthermore, this method has a problem in which it is not possible to perform light irradiation on cells and observation of cells at the same time. Therefore, a method capable of selectively sorting cells, while observing the cells in the container with the image analyzing apparatus and grasping the coordinate position and state of the cells, is required.

### BRIEF DESCRIPTION OF THE DRAWINGS

FIG. 1 is a schematic view of a cell selecting system according to an embodiment;

FIG. 2A and FIG. 2B is a view of a cell selecting apparatus according to the embodiment;

FIG. 3 is a characteristic view illustrating the light absorption wavelength dependency of a DNA in a cell;

FIG. 4A and FIG. 4B is a view of the cell selecting apparatus according to the embodiment; and

FIG. 5A and FIG. 5B is a view of the cell selecting apparatus according to the embodiment.

### DETAILED DESCRIPTION

Embodiments of the invention will be described below with reference to the drawings.

Parts with the same reference numerals indicate similar parts.

The drawings are schematic or conceptual, and a relation between the thickness and the width of each portion, a ratio coefficient of the size between the portions, and the like are not necessarily the same as the actual ones. Even when representing the same part, there are cases where the dimensions or ratio coefficients are different from each other depending on the drawings.

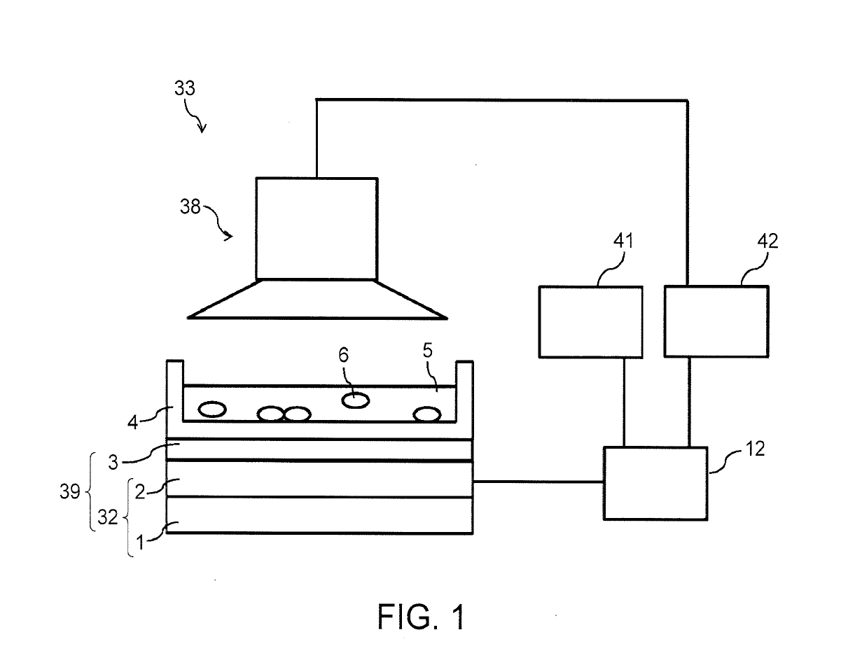

FIG. 1 illustrates a schematic view of the cell selecting system according to the embodiment.

A cell selecting system 33 includes a cell selecting apparatus 39, a container 4, a controller 12, an imaging apparatus 38, an input unit 41, and a display unit 42. The container 4 for holding a cell sample, a culture solution and the like is placed on the cell selecting apparatus 39. The container 4 is a cell culture container.

The cell selecting apparatus 39 has a light source device 32 and a stage 3.

The light source device 32 has a substrate 1 and a light source group 2. The light source group 2 is located above the substrate 1. The substrate 1 supports the light source group 2. The light source group 2 includes a plurality of light source elements set in the matrix array. Each light source element is at least an element capable of emitting light having an operation of damaging a deoxyribonucleic acid (DNA) in a cell, such as ultraviolet rays.

The stage 3 is located above the light source group 2 of the light source device 32 and is provided for placing the container 4 (culture substrate). In order to transmit the light from the light source group 2 to the container 4, the stage 3 is formed of a light transmissive material such as glass, quartz, transparent resin or the like, or may be a light transmissible mesh, mesh body, or may be a frame structure which supports the container 4.

The container 4 is placed on the stage 3. FIG. 1 illustrates a state in which the culture solution 5 and a plurality of cells 6 are contained in the container 4. While the material of the container 4 may be formed of a light transmitting material, for example, glass, quartz, resin, or the like, from the viewpoint of ease of transmission of ultraviolet light, it is preferable that the container be formed of quartz.

The controller 12 is connected to the light source device 32 of the cell selecting apparatus 39. The controller 12 controls light emission of the light source group 2 of the light source device 32.

The imaging apparatus 38 is disposed above the container 4. The imaging apparatus 38 is a microscope equipped with an imaging device. An operator of the cell selecting system 33 uses the imaging apparatus 38 and the display unit 42 connected thereto to observe the inside of the container 4 from the upward direction of the container 4 and to grasp (understand) the coordinate position and the state of the cells 6. A mechanism in which the imaging apparatus automatically recognize the cells by using an image recognition technology may be adopted.

The input unit 41 is connected to the controller 12. The input unit 41 transfers various instructions and various types of setting information, which have been input by operating a mouse, a keyboard or the like through the operator, to the controller 12. The input unit 41 accepts designation or the like of the light source element that emit light in the light source group 2, from the operator.

The display unit 42 is connected to the controller 12 and the imaging apparatus 38. The display unit 42 is a monitor device referred to by an operator. Under the control of the controller 12, the display unit 42 displays the cells 6 in the container 4 captured by the imaging apparatus 38. The display unit 42 displays various instructions from the operator via the input unit 41.

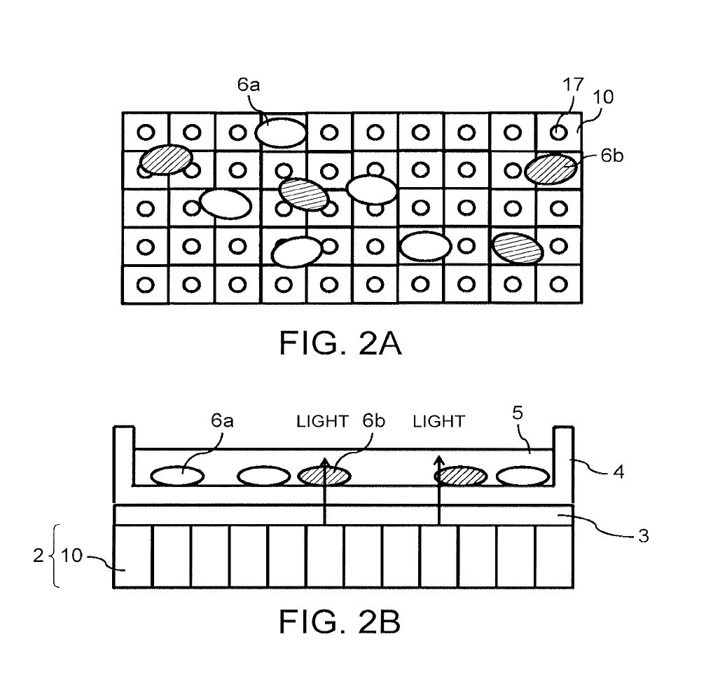

FIG. 2A is a plan view of the container 4 of the cell selecting system 33 and the light source group 2 located thereunder when viewed from above, and FIG. 2B is a cross-sectional view of the light source group 2 and the container 4 of the cell selecting system 33.

Hereinafter, a method of using the cell selecting system of the embodiment will be described.

FIG. 2A illustrates a state in which normal cells 6a and unnecessary cells 6b are present in the container 4. In the light source group 2, a plurality of light source elements 10 is set in the matrix array. Each light source element 10 has an opening portion 17, and light is extracted from the opening portion 17.

The operator observes the cells in the container 4 by the image captured by the imaging apparatus 38 to recognize the normal cells 6a or the unnecessary cells 6b and grasp the respective positions of the normal cells 6a and the unnecessary cells 6b.

The unnecessary cells 6b include, for example, cells in which the normal cells 6a or other cells are mutated to cancer cells or the like. The unnecessary cells are not limited thereto. When the operator selects only the normal cells 6a from the normal cells 6a and unnecessary cells 6b, the operator inputs the positions of the normal cells 6a or the unnecessary cells 6b to the input unit 41 connected to the controller 12. The position information of each of the normal cells 6a and the unnecessary cells 6b is transmitted to the controller 12.

Thus, in the light source device 32 of the cell selecting system 33, as illustrated in FIG. 2B, it is possible to cause the specific light source element 10 corresponding to the position of the unnecessary cell 6b of the light source group 2 to emit light. When the light source element 10 emits light to the unnecessary cell 6b, light enters the unnecessary cell 6b, and the DNA of the unnecessary cell 6b is damaged. The unnecessary cell 6b in which DNA is damaged dies or does not proliferate (multiply, grow).

Meanwhile, since the light emitting element 10 located at the position of the normal cell 6a does not emit light, the normal cell 6a does not receive the light emission of the light source element 10. The normal cells 6a can survive and maintain a proliferous state.

Therefore, in the cell selecting system 33 of the embodiment, it is possible to selectively destroy (kill) only the unnecessary cell 6b or inhibit the proliferation, and to make only the normal cells 6a present in the container 4 and select the normal cells 6a.

In this system, since it is unnecessary to move the normal cells 6a and the unnecessary cells 6b to separate containers after acquiring the coordinate positions of the cells in the process of selecting the normal cells 6a, the position information of the normal cells 6a and the unnecessary cells 6b does not lost. Therefore, it is possible to select the normal cells 6a, while checking the successive change in the proliferation of the normal cells 6a.

Although a manual embodiment in which the operator inputs the position information of the unnecessary cell 6b to the input unit 41 has been illustrated, the image pickup device 38 may automatically recognize the unnecessary cell 6b, and the controller 12 may automatically control the light emitting element 10 corresponding to the existing position of the unnecessary cells 6b to emit light.

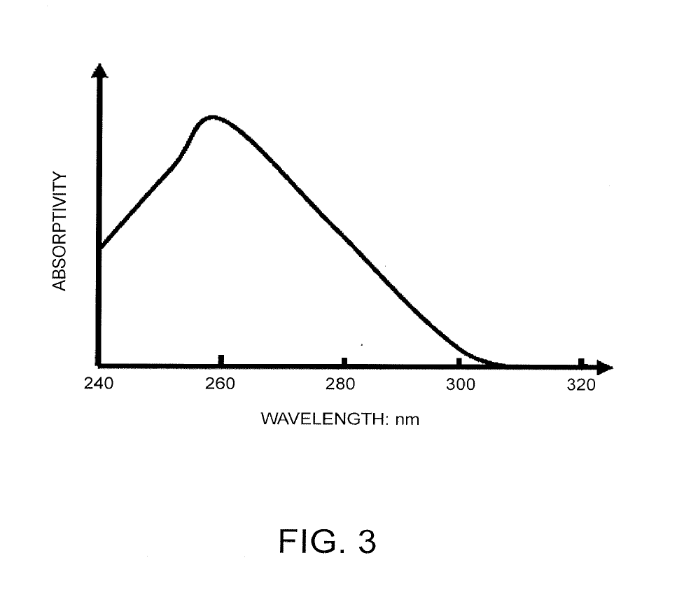

FIG. 3 illustrates a relation of the light absorption wavelength dependency of the DNA present inside the cell. A horizontal axis is the wavelength, and a vertical axis is the absorptivity.

The DNA present inside the normal cells 6a and the unnecessary cells 6b most absorbs ultraviolet light having a wavelength of about 260 nm. That is, the DNA present inside the normal cells 6a and unnecessary cells 6b tends to be damaged when irradiated with ultraviolet light having a wavelength of 260 nm. Therefore, the wavelength of the light emitted from the light source element 10 of the cell selecting system 33 is preferably about 260 nm, for example, 200 nm or more and 300 nm or less.

The cell selecting system 33 will be described in more detail below.

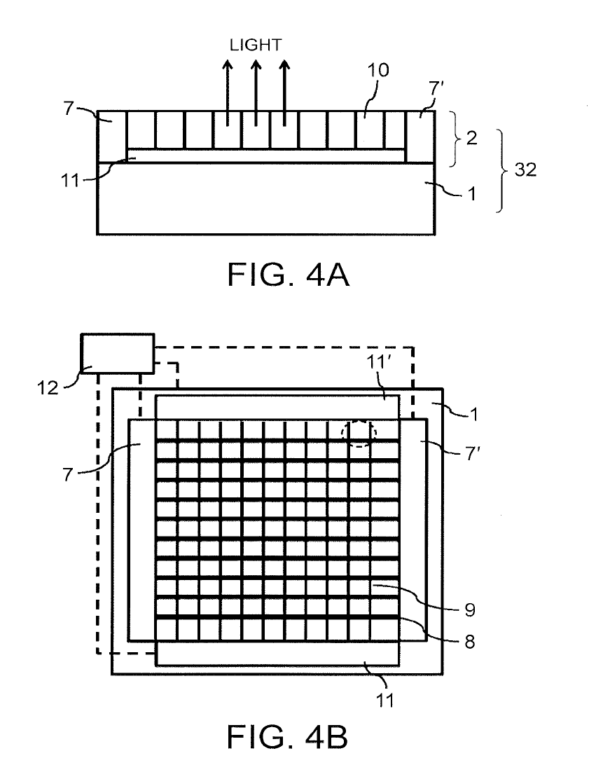

FIG. 4 illustrates a schematic view of the light source device 32 of the cell selecting system 33.

FIG. 4A is a side sectional view of the light source device 32 of the cell selecting system 33. As illustrated in FIG. 4A, in the light source device 32, the light source group 2 is arranged on the substrate 1. The light source group 2 has a plurality of light source elements 10. Individual light source elements 10 constituting the light source group 2 are light emitting diodes (LEDs). The plurality of light source elements 10 is disposed to emit light in a direction opposite to the substrate 1, that is, toward the container 4. The light emitting diode of the light source element 10 emits at least ultraviolet light, for example, light having a wavelength of 200 nm or more and 300 nm or less. In addition to the light emitting diode that emits ultraviolet light as the light source element 10, a light emitting element which emits light of another wavelength, such as a light emitting diode which generates visible light suitable for other uses such as cell observation, may be arranged in combination. The visible light source for cell observation may be provided above the container 4, but may be provided on the light source group 2 side. A light emitting diode that generates visible light may be provided in each light source element together with a light emitting diode that generates ultraviolet light, or may be arranged by being dispersed in the light source group 2.

Further, the light source device 32 includes two address line drive units 7 and 7′ and two data line drive units 11 and 11′ (11′ is not illustrated). As it will be described later, each light source element 10 is driven, by applying a voltage to a plurality of address lines and a plurality of data lines connected to the address line drive units 7 and 7′ and the data line drive units 11 and 11′.

FIG. 4B is a plan view of the light source device 32 of FIG. 4A.

As illustrated in FIG. 4B, two address line drive units (wiring drive units) 7 and 7′, an address line 8 (first wiring), a data line 9 (second wiring), and two data line drive units (wiring drive units) 11 and 11′ are disposed on the substrate 1.

On the substrate 1, the two address line drive units 7 and 7′ are spaced apart and arranged to face each other. A plurality (for example, m (m>1)) of address lines 8 are disposed between the two address line drive units 7 and 7′, and are connected to the address line drive units 7 and 7′, respectively. Each of the plurality of address lines 8 is arranged substantially in parallel with each other.

Further, on the substrate 1, the two data line drive units 11 and 11′ are spaced apart and arranged to face each other. The two data line drive units 11 and 11′ are arranged in a direction substantially perpendicular to the direction in which the two address line drive units 7 and 7′ are arranged. A plurality of (for example, n (n>1)) data lines 9 are arranged between the two data line drive units 11 and 11′, and is connected to the data line drive units 11 and 11′. Each of the plurality of data line sections 9 is arranged substantially in parallel with each other. That is, the address line 8 and the data line 9 intersect with each other in the form of an m×n matrix.

The controller 12 is connected to each of the address line drive units 7 and 7′ and the data line drive units 11 and 11′ by wirings (the wirings are indicated by a dotted line in FIG. 4B).

The controller 12 controls the address line drive units 7 and 7′ and the data line drive units 11 and 11′ in order to apply a voltage to the address line 8 and the data line 9. The light source element 10 emits light when a voltage is applied to the address line 8 and the data line 9. The voltage of the address line 8 is controlled by the controller 12 via the address line drive units 7 and 7′. The voltage of the data line 9 is controlled by the controller 12 via the data line drive units 11 and 11′. In FIGS. 4A and 4B, the positions of the address line drive units 7 and 7′ and the data line drive units 11 and 11′ may be reversed.

Here, a case where the light source element 10 emits light at a voltage equal to or higher than a certain threshold value Vₜₕ (V) will be considered. For example, a voltage of V₁ (V) (or 0 V) is applied to the y-th data line 9 from the right of FIG. 4B, and a voltage of 0 V (or V₂ (V)) is applied to the x-th address line 8 from the top of FIG. 4B. In this case, when the relation of “Vₜₕ (V)<applied voltage (V) of the light source element” is satisfied, the light source element 10 at the position of y-row x-column can be caused to selectively emit light. When the light source element is an LED, the light source element emits light with forward bias of the threshold value Vₜₕ (V) or more.

Besides, the circuit may be configured so that the light source element 10 emits light at the time of Vₜₕ (V)<V₁+V₂ (V). In this case, each of the voltages V₁ (V) and V₂ (V) is a positive value. If the relations of Vₜₕ (V)<V₁ (V) and Vₜₕ (V)<V₂ (V) are satisfied, the light source elements 10 other than the y-row and the x-column do not emit light.

By applying voltages to the address line 8 and the data line 9, it is possible to cause each light source element 10 of the (m, n) matrix arrangement, in which the m light source elements 10 are arranged in the arrangement direction of the data line section 9 and m light source elements 10 are arranged in the arrangement direction of the address line section 8, to arbitrarily emit light.

Two or more light source elements 10 can be selectively caused to emit light as well as one light source element.

Further, in this embodiment, a passive matrix (simple matrix) driving method was used as a method of applying a voltage to the address line 8 and the data line 9. In the invention, it is not limited to the passive matrix driving method. For example, the method is also applicable to other driving method such as an active matrix driving method in which a transistor is connected to each light source element 10 in order to select the light source element 10.

Next, the light emitting element 10 will be further described.

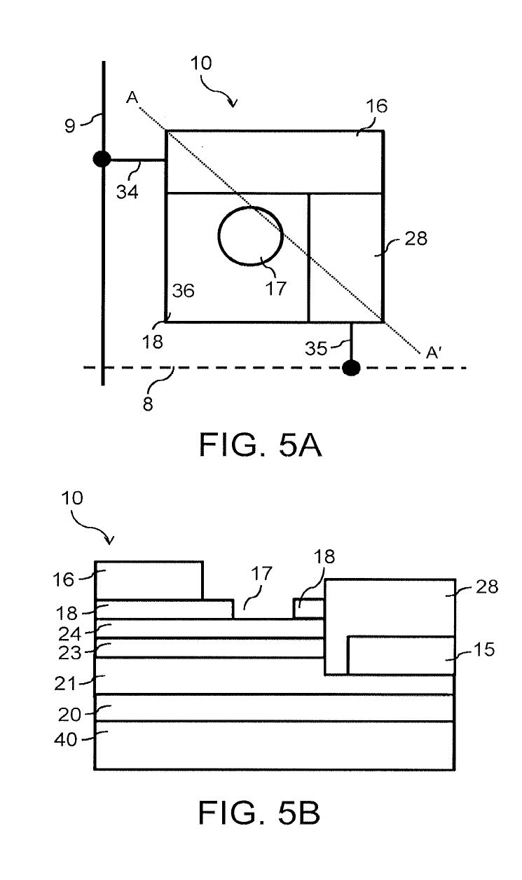

FIG. 5A illustrates an enlarged plan view of the light source element 10 constituting a light source group 2. FIG. 5A is an enlarged plan view of a part surrounded by a dotted circle frame of FIG. 4B, and is a view of the light source element 10 viewed from the top of the light emitting device 33.

The address line 8 is electrically connected to the light source element 10 via a wiring 35. The data line is connected to the light source element 10 via the wiring 34. The address line 8 and the data line 9 intersect with each other, but are not connected to each other. In the case of FIG. 5A, the address line 8 is in the depth direction of the sheet compared to the data line 9. Therefore, the address line 8 is indicated by a dotted line and the data line 9 is indicated by a solid line.

FIG. 5B is a cross-sectional view taken along line A-A′ of the light emitting element 10.

The dotted line A-A′ illustrated in FIG. 5A illustrates the position of the cross section of the cross-sectional view of the light source element 10 illustrated in FIG. 5B.

The light emitting element 10 includes a support substrate 40, a first semiconductor layer 20, a second semiconductor layer 21, a third semiconductor layer 23, a fourth semiconductor layer 24, an upper electrode layer 18, a first electrode 15, a second electrode 16, an insulating protective layer 28, and an opening portion 17.

The first semiconductor layer 20 is provided on the support substrate 40. The first semiconductor layer 20 is a buffer layer.

The second semiconductor layer 21 is provided on the first semiconductor layer 20. The second semiconductor layer 21 is, for example, an n-type GaN layer.

The third semiconductor layer 23 is provided on the second semiconductor layer 21. The third semiconductor layer 23 is, for example, an active layer in which an AlGaN layer and a GaN layer are stacked.

The fourth semiconductor layer 24 is provided on the third semiconductor layer 23. The fourth semiconductor layer 24 is, for example, a p-type GaN layer.

The upper electrode layer 18 is provided on the fourth semiconductor layer 24. The upper electrode 18 is, for example, a p-type electrode or a metal electrode. The upper electrode layer 18 has the opening portion 17.

The first electrode 15 is provided on the second semiconductor layer 21. The first electrode 15 is, for example, an n-type electrode or a metal electrode.

The second electrode 16 is provided on the upper electrode layer 18. The second electrode 16 is, for example, a p-type electrode or a metal electrode.

The first electrode 15 is covered with the insulating protective layer 28 to ensure insulation between the first electrode 15 and the second electrode 16.

By changing the composition and thickness of each of the first semiconductor layer 20, the second semiconductor layer 21, the third semiconductor layer 23, and the fourth semiconductor layer 24, the wavelength of the light emitted from the light source element 10 can be changed.

Here, the first electrode 15 is connected to the address line 8, and the second electrode 16 is electrically connected to the data line 9. In the light source element 10, a semiconductor layer emits light by applying a desired voltage between the first electrode 15 and the second electrode 16, and light is extracted from the opening portion 17.

In the light source element 10, light is extracted from the opening portion 17. The opening portion 17 is provided on the upper electrode layer 18. The opening portion 17 has a circular shape. It is desirable that the area of the opening portion 17 be 50% or less of the area of the light-emitting side of the light source element 10, that is, the area of the upper surface portion of the light source element 10. The shape of the opening portion 17 is not limited to a circular shape, but may be, for example, a square shape.

The plane arrangement of the light source element 10 from the upper part is disposed so that the second electrode 16, the upper electrode layer 18, the opening portion 17, and the insulating protective layer 28 are disposed on the upper surface, as illustrated in FIG. 5A.

According to the embodiment, a cell selecting apparatus and a cell selecting system capable of selecting a desired cell, while grasping the coordinate position and state of the cell in the container.

In the cell selecting apparatus and the cell selecting system of the embodiment, a cell sample is placed on the light source group, the light emitting element at a position corresponding to the unnecessary cell is selectively caused to emit light, the unnecessary cell is damaged by entrance of light into the unnecessary cell, thereby destroying or not proliferating the abnormal cell, and normal cells are kept in a proliferous state.

As the cells, cells of living things such as human body can be targeted.

The cell selecting apparatus and the cell selecting system can be called as cell selecting apparatus and cell selecting system, and also called as cell collecting apparatus and cell collecting system.

While several embodiments of the invention have been described, these embodiments have been presented by way of example and are not intended to limit the scope of the invention. The embodiments can be provided in various other forms, and various omissions, substitutions, and changes can be made without departing from the gist of the invention. The embodiments and the modifications are included in the scope and gist of the description as well as the invention described in the claims and the equivalent scope thereof.

What is claimed is:

1. A cell selecting apparatus comprising:
a light source device for placing a cell sample, the light source device including a plurality of light source elements arranged in a matrix form.

2. The cell selecting apparatus according to claim 1,
wherein the light source device includes:
a substrate;
a plurality of first wirings arranged on the substrate;
a plurality of second wirings arranged to intersect with the plurality of first wirings in a matrix form; and
a plurality of light source elements provided for each intersection between the plurality of first wirings and the plurality of second wirings, and connected to the first wirings and the second wirings.

3. The cell selecting apparatus according to claim 2,
wherein the light source device includes:
a wiring drive unit connected to the plurality of first wirings and the plurality of second wirings; and
a processor connected to the wiring drive unit to control driving of each of the plurality of light emitting elements.

4. The cell selecting apparatus according to claim 1, further comprising a stage disposed on the light source device,
transmitting light from the plurality of light emitting elements, and the stage is used for placing a container.

5. The cell selecting apparatus according to claim 1, wherein each of the plurality of light source elements emits ultraviolet light.

6. The cell selecting apparatus according to claim 1, wherein each of the plurality of light source elements emits light in a wavelength of 200 nm or more and 300 nm or less.

7. The cell selecting apparatus according to claim 2, wherein the plurality of first wirings is an address line and the plurality of second wirings is a data lines.

8. The cell selecting apparatus according to claim 1, wherein the light source element has an opening portion on an electrode layer.

9. The cell selecting apparatus according to claim 1,
wherein the light source element includes:
a first conductivity semiconductor layer;
a second conductivity semiconductor layer located on the first conductivity semiconductor layer;
a first electrode located on the first conductivity semiconductor layer;
an upper electrode layer located on the second conductivity semiconductor layer;
a second electrode located on the upper electrode layer; and
an opening portion provided on the upper electrode layer of the light source element.

10. The cell selecting apparatus cording to claim 1, wherein the light source element has an active layer between the first conductivity semiconductor layer and the second conductivity semiconductor layer.

11. A cell selecting system comprising:
the cell selecting apparatus according to claim 1;
an imaging apparatus disposed above the cell selecting apparatus;
a display unit displaying an image captured by the imaging apparatus; and
an input unit inputting an instruction to the cell selecting apparatus.

12. The cell selecting system according to claim 11, wherein the imaging apparatus includes a microscope.

13. The cell selecting system according to claim 11, wherein the light emitting element at a position corresponding to an unnecessary cell is caused to emit light.
